(19) United States
(12) Patent Application Publication
(10) Pub. No.: US 2008/0257637 A1
(43) Pub. Date: Oct. 23, 2008

(54) Disposable Stethoscope Head Cover and Automated Dispensing Apparatus Therefor

(76) Inventors: Bruce D. Miller, N. Reading, MA (US); Diana Pehlman-Wilkin, Plano, TX (US); C. Scott Wilkin, Plano, TX (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/737,370

(22) Filed: Apr. 19, 2007

(51) Int. Cl. A61B 7/02

(52) U.S. Cl. 181/131

(57) ABSTRACT

A method and apparatus for the prevention of carryover infection due to surface contamination on a stethoscope features a disposable stethoscope head cover implemented with hypoallergenic materials and a dispensing apparatus that automatically places a disposable stethoscope head cover in an attachment station, allowing a simple single handed attachment of the disposable stethoscope head cover to the stethoscope.

### BACKGROUND

The acoustic stethoscope has been a primary diagnostic tool for clinicians and physicians for many years. It is used for diagnosing abnormalities of pulmonary and cardiac function based upon auscultation. The acoustic stethoscope comprises a passive acoustic amplifier based upon a sound chamber with a diaphragm or an acoustic bell. Electronic versions of the stethoscope have been developed with signal processing to mimic the acoustic characteristics of the passive acoustic stethoscope. These stethoscopes are in the market, albeit not as widely used as the acoustic stethoscope.

A persistent issue with the stethoscope (acoustic and electronic) is the potential for infectious agents to be spread from patient to patient (and clinician). The stethoscope must be placed on bare skin for it to work properly and that carries the risk of infectious agent transfer from the skin to the stethoscope and then to other patients (or clinician). Standard clinical practice involves cleaning the stethoscope between patients using a disinfectant agent. However, this is cumbersome and time consuming. As such, it is not always done. Over the years, several approaches have been developed to address this problem. None of them have become widely accepted in the marketplace. All of these approaches have issues that have precluded their widespread acceptance. In many cases, their use is cumbersome and eschewed by clinicians/physicians. In most medical practice, time and efficiency are critical and devices/techniques must be efficient and expedient. In addition, some of the approaches use materials that have been found to cause severe allergic reactions (such as latex rubber).

### SUMMARY

There is a need for a method and apparatus to prevent carryover infection based upon physical contact with the head of a standard stethoscope (acoustic or electronic). In accordance with the present invention, a disposable element and a dispensing apparatus in combination provide a system that provides both the protection that the clinical community desires and the ease of use necessary for widespread adoption. Embodiments of the disposable element can be manufactured inexpensively using hypoallergenic materials such as vinyl.

In one aspect, a disposable stethoscope head cover comprises a hypoallergenic membrane member configured to be secured to a stethoscope head to inhibit physical contact between the skin of a patient and the stethoscope head, and a support member configured to hold the membrane member to the stethoscope head. The hypoallergenic membrane member can be made of vinyl. The support member can be a fastening ring, can be made of a rigid material selected from the group consisting of plastic, rubber and nylon, and can be configured to avoid contact with the patient during use. The disposable stethoscope head cover can further comprise a removal tab facilitating removal of the disposable stethoscope head cover from the stethoscope head.

In another aspect, an automated dispensing apparatus comprises a pre-staging shelf, a feeder tube vertically disposed above the pre-staging shelf and configured to hold plural items wherein an item to be dispensed is fed into the pre-staging shelf, a staging station, a dispensing arm configured to push the item to be dispensed from the pre-staging shelf to the staging station, and means for moving the dispensing arm. The automated dispensing apparatus can further comprise a light source and a photo detector to detect an empty condition of the pre-staging shelf, a sensor to detect the presence of the feeder tube, a light source and a photo detector to indicate whether the item to be dispensed is positioned on the staging station, a position tab and a plurality of photo detectors to detect home and dispensed positions of the dispensing arm, or a guide member wherein the dispensing arm is translated across the guide member, or combinations thereof. The means for moving the dispensing arm can be a wheel attachable to a linkage attachable to the dispensing arm, or a drive gear, and can be driven by a stepper motor. The automated dispensing apparatus can be used to dispense a disposable stethoscope head cover.

In yet another aspect, a method of preventing cross patient exposure to infectious agents due to stethoscope carryover comprises disposing a hypoallergenic membrane over a surface of the stethoscope head on one surface of the membrane, holding the membrane to the stethoscope head with a rigid support, physically contacting a patient on the other surface of the membrane during examination with the stethoscope and removing the membrane and the rigid support from the stethoscope head after examination. The hypoallergenic membrane can be made of vinyl. The rigid support can be a fastening ring or made of a rigid material such as plastic, rubber or nylon, and can be configured to avoid contact with the patient during use. Removal of the membrane and the rigid support from the stethoscope head can be facilitated by a removal tab.

In still another aspect, a method of dispensing an item to be dispensed, wherein the item can be a disposable stethoscope head cover, comprises feeding the item to be dispensed from a feeder tube into a pre-staging shelf and pushing the item to be dispensed with a dispensing arm from the pre-staging shelf into position in a staging station. The method can further comprise detecting an empty condition of the pre-staging shelf with a light source and a photo detector, detecting the presence of the feeder tube with a sensor, detecting whether the item to be dispensed is positioned on the staging station with a light source and a photo detector, or detecting home and dispensed positions of the dispensing arm with a position tab and a plurality of photo detectors, or combinations thereof.

### BRIEF DESCRIPTION OF THE DRAWINGS

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1 is a perspective view of a stethoscope head.

FIG. 2A illustrates a middle cross-section view of a disposable stethoscope head cover in isolation.

FIG. 2B illustrates a middle cross-section view of a disposable stethoscope head cover attached to a stethoscope head.

FIG. 2C illustrates a perspective view of a fastening ring.

FIG. 3A is a side view of an embodiment of an automated dispensing apparatus in home position.

FIG. 3B is a side view of an embodiment of an automated dispensing apparatus in dispensed position.

FIG. 4 is a top view of the automated dispensing apparatus of FIGS. 3A and 3B.

FIG. 5 is a side view of an alternate embodiment of an automated dispensing apparatus using a rack/pinion drive mechanism.

FIG. 6 is a diagram of drive circuitry for a bipolar permanent magnet stepping motor for use in the preferred embodiments of the automated dispensing apparatus of FIGS. 3A, 3B, 4 and 5.

FIG. 7 is a block diagram of an embodiment of electronics for use in the automated dispensing apparatus of FIGS. 3A, 3B, 4 and 5.

FIG. 8 is a block diagram of an operating system and routines for implementing control and operation of the automated dispensing apparatus of FIGS. 3A, 3B, 4 and 5.

FIG. 9 is a block diagram of an initialization routine.

FIG. 10 is a block diagram of a dispensing routine.

### DETAILED DESCRIPTION

A description of example embodiments of the invention follows.

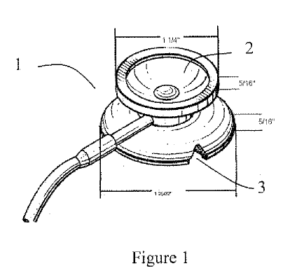

The present invention is directed toward the prevention of carryover infection between patients (and clinicians) due to physical contact with the head of a stethoscope (acoustic or electronic). FIG. 1 shows the head of a standard double sided acoustic stethoscope. The stethoscope head 1 provides the physical contact point to the patient to allow the clinician to hear cardiac or pulmonary auscultation during an examination. The head of the stethoscope contacts the skin of the patient to allow the sound to travel through the skin and couple to the stethoscope. In a double sided stethoscope, one side of the head 1 is comprised of a smooth surface 3 with an internal diaphragm. The diaphragm is used to couple the sound from the patient to an acoustic tube leading to the earpiece of the stethoscope (not shown). The opposite side of the stethoscope comprises a concave chamber 2 which forms an acoustic compartment (when coupled to the skin) and directs sound to the acoustic tube leading to the earpiece of the stethoscope (not shown). The clinician can use either side of the stethoscope during a physical examination based upon preference. The presentation of the coupled sound is different based upon the two methods as they have different acoustic properties. Both methods have been widely used historically and there are interpretive preferences among clinicians in the market.

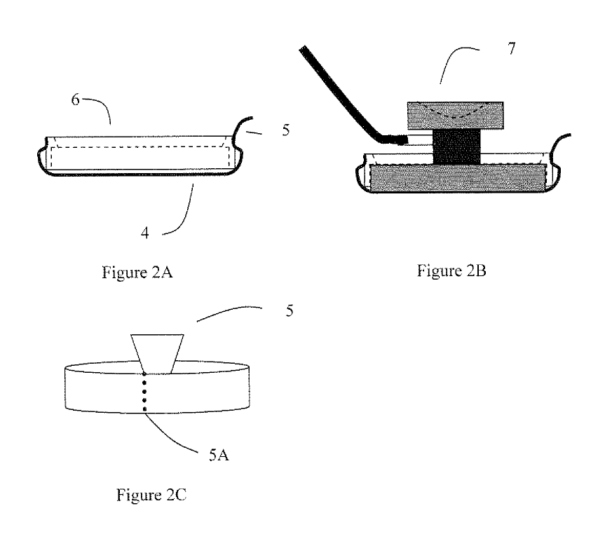

FIG. 2A shows an embodiment of a disposable stethoscope head cover in isolation. The disposable stethoscope head cover in the preferred embodiment comprises a vinyl membrane 4. The vinyl membrane physically contacts the patient on the bottom surface of the shown view. Vinyl is a preferred material for hospital use based upon its hypoallergenic properties, though other suitable materials may be used. The vinyl membrane 4 is attached to a fastening ring 6. The fastening ring is made of plastic or rubber or nylon or another rigid material with limited elasticity and provides a rigid support to hold the vinyl membrane to the head of the stethoscope while allowing the stethoscope head to be attached and removed easily. The fastening ring does not come into contact with the patient or the clinician during use. FIG. 2C shows a fastening ring. A removal tab 5 attached to the fastening ring can be provided to facilitate removal of the disposable stethoscope head cover from the stethoscope head. The fastening ring call include a perforated line 5A to facilitate the removal process. FIG. 2B shows the disposable stethoscope head cover attached to one side of the head of a dual sided acoustic stethoscope 7. In this particular example the disposable stethoscope head cover is shown attached to the diaphragm side of the stethoscope head. Alternatively, the concave bell side may be accommodated with a differently dimensioned disposable stethoscope head cover. The vinyl membrane 4 is stretched over the surface of the stethoscope head (in tension) when the disposable stethoscope head cover is attached. The stretching of the vinyl membrane insures a tight coupling between the skin and the acoustic stethoscope head. The vinyl membrane is flexible and will transfer sounds without changing the acoustic information when stretched.

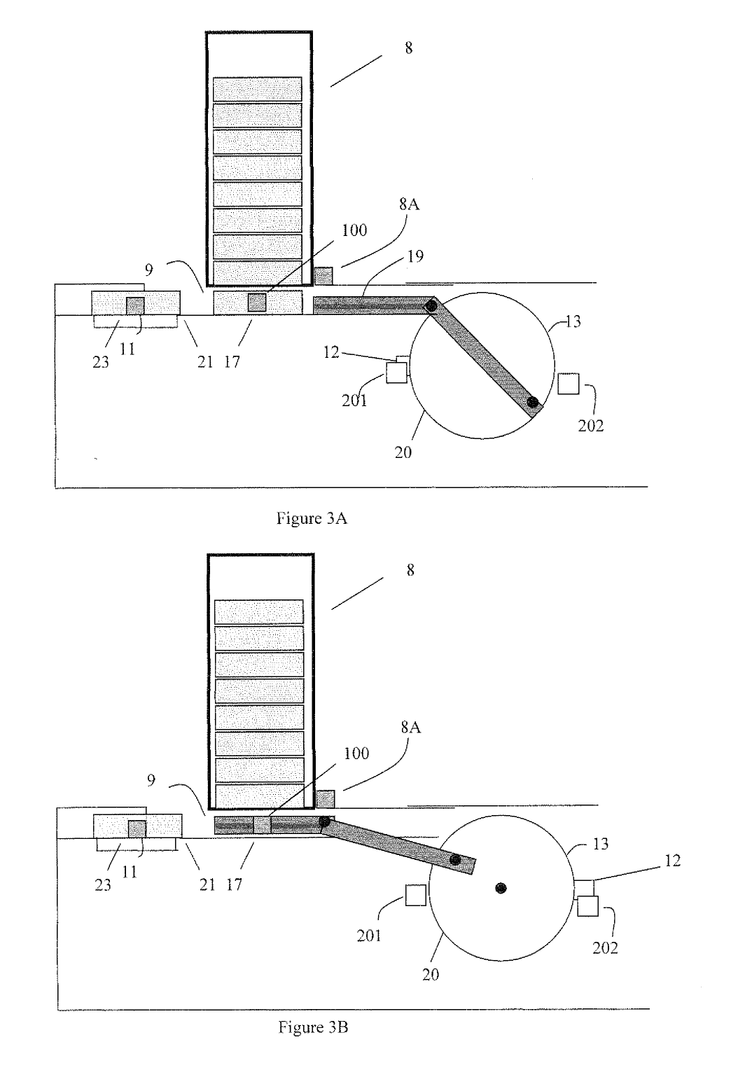

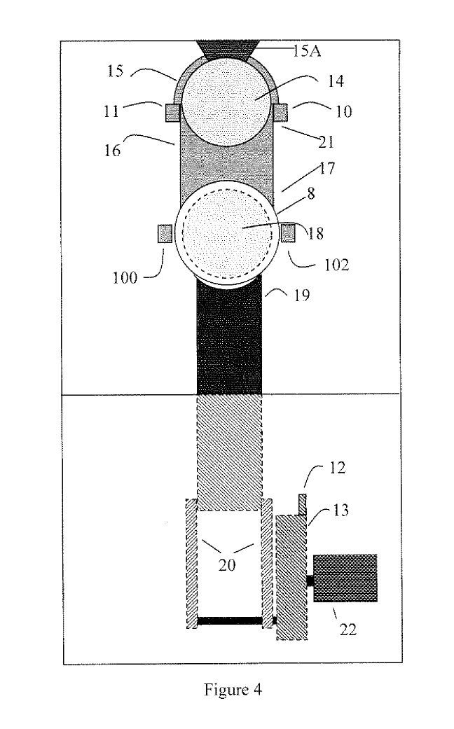

FIGS. 3A, 3B and 4 show an embodiment of an automated dispensing apparatus for a disposable stethoscope head cover. While the item to be dispensed in the embodiment is a disposable stethoscope head cover, it will be understood by those skilled in the art that the automated dispensing apparatus of the present invention can be used to dispense any item of interest, provided that certain changes in form and details be made without departing from the scope of the invention.

The disposable stethoscope head cover 9 is packaged in a feeder tube 8 with the vinyl membrane at the bottom. The feeder tube is opened and inserted into the dispensing apparatus so that the disposable stethoscope head covers can be one-by-one gravity fed into a pre-staging shelf 17 on the dispensing apparatus with the vinyl membrane at the bottom and stand ready for dispensing. Alternatively, the feeder tube can comprise means for forcing the disposable stethoscope head cover into the pre-staging shelf. An example of such means for forcing the disposable stethoscope head cover into the pre-staging shelf is a spring. A second disposable stethoscope head cover is shown in an attachment station 21 (also referred to as staging station) with the vinyl membrane at the bottom and is ready for use. The center of the attachment station has a recess 23. The area of the recess is smaller than that encompassed by the fastening ring of the disposable stethoscope head cover so that the fastening ring can be supported by the edge of the recess and so that the disposable stethoscope head cover does not fall into the recess. The area of the recess is larger than the surface area of the stethoscope head, so as to allow the stethoscope head to move downward into the recess for the disposable stethoscope head cover to snap into place. The stethoscope head is pressed downward into the disposable stethoscope head cover until it snaps into place. This can be done with a single hand and an efficient motion. A photo detector 11 can be used in conjunction with a light source 10 (FIG. 4) to indicate whether the disposable stethoscope head cover is positioned on the attachment station. When running in an automatic mode, the dispensing apparatus can automatically dispense a disposable stethoscope head cover upon detection of its absence in the attachment station, so that a disposable stethoscope head cover is always kept in the attachment station 21 ready for use. A stepper motor 22 (FIG. 4) can be used to drive a wheel 13. A position tab 12 can be used in conjunction with photo detectors 201 and 202 to detect home position (as shown in FIG. 3A) and dispensed position (as shown in FIG. 3B) of a dispensing arm 19.

The rotation of the drive wheel 13 is translated into horizontal motion based upon movement of the dispensing arm 19 moving horizontally in a track or a guide. During a dispensing cycle, a new disposable stethoscope head cover 9 is pushed from the pre-staging shelf 17 into the attachment station 21 by the dispensing arm 19. When the dispensing arm 19 is retracted, a new disposable stethoscope head cover drops into position in the pre-staging shelf. A light source 100 and a photo detector 102 (FIG. 4) are used to detect an empty condition of the pre-staging shelf 17. The empty condition occurs when the dispensing arm 19 is in home position (as shown in FIG. 3A) and there is no disposable stethoscope head cover in the pre-staging shelf 17.

A sensor 8A can be used to detect the presence of the feeder tube 8. If the feeder tube 8 is not inserted, it is empirically known that the system is in the empty state although there could be a disposable stethoscope head cover in the pre-staging shelf waiting to be dispensed or in the attachment station ready for use. When a feeder tube is inserted, the dispensing apparatus can, facilitated by software to be described later, keep a count of the number of dispensed disposable stethoscope head covers and therefore know how many disposable stethoscope head covers remain in the feeder tube. This allows the system to provide an early indication that a new feeder tube will be needed. The clinician does not have to wait for the system to be empty to collect a new feeder tube. In some cases, it may not be known how many disposable stethoscope head covers remain in the feeder tube.

Note that the diameters of the feeder tube and of the recess in different automated dispensing apparatus may vary to accommodate differently dimensioned disposable stethoscope head covers.

FIG. 4 shows the automated dispensing apparatus as viewed from the top. The attachment station 21 is shown with a disposable stethoscope head cover 14 in position above recess 23 for attachment to a stethoscope head. The photo detector 11 and light source 10 are shown on the sides of a detent 15. The detent on the top most side of the attachment station is cut back, and the resulting gap 15A allows the clinician to attach the disposable stethoscope head cover without interference with the stethoscope's acoustic tube. The disposable stethoscope head cover sits in position with the fastening ring supported by the edge of the recess while the stethoscope head stretches the membrane and snaps the fastening ring into place in a downward movement into the recess. The feeder tube 8 is shown with a disposable stethoscope head cover 18 in position for dispensing. The light source 100 in coordination with the photo detector 102 can be used to detect an empty condition based upon no disposable stethoscope head cover in the pre-staging shelf 17 when the dispensing arm 19 is in home position. The dispensing arm 19 can push the disposable stethoscope head cover forward, across a guide 16 and into position in the attachment station against the detent 15. The dispensing arm 19 can be attached to a linkage 20 attachable to the wheel 13 attachable to the stepper motor 22. The position tab 12 can be used to detect home position and dispensed position, as previously described. The position of the linkage 20 on the wheel 13, along with its length, can determine the overall length of travel of the dispensing arm 19, the speed and the relative mechanical moment.

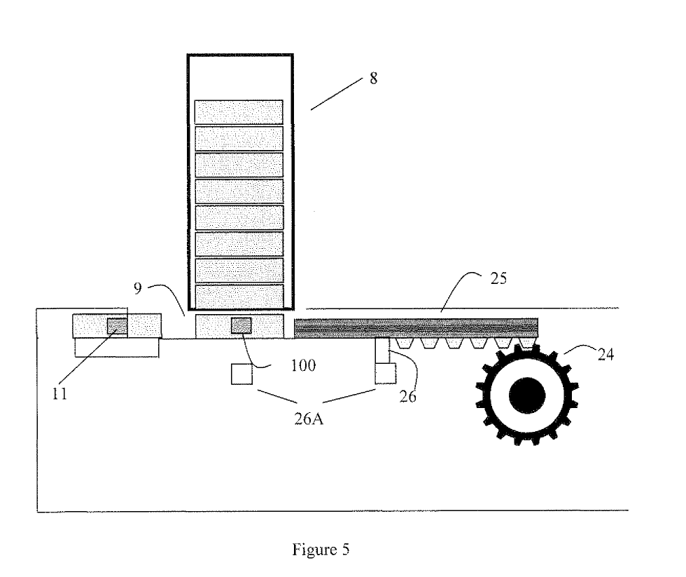

FIG. 5 shows an alternative embodiment of an automated dispensing apparatus using a rack and pinion drive system. A drive gear 24 can be used to move the dispensing arm 25 (also called a rack). A position tab 26 can be used to detect home and dispensed positions of the dispensing arm 25 using a pair of photo detectors 26A. By using a stepper motor, the position of the rack relative to home and dispensed positions can be known based upon the number of steps that the stepper motor takes. The angular rotation of a step is translated into the linear motion of the rack and the number of steps that the stepper motor takes can represent the distance that the rack travels, provided that the rack does not go off the drive gear. In the event that the stepper motor takes more than the requisite number of steps (by some margin), then it can be known that some sort of malfunction (such as a jam) has occurred. The speed and torque applied to the dispensing arm 25 can be determined by the gear size and the stepper motor. In this example the gear is shown with very coarse teeth; in practice, much finer toothed gear can be used to provide smooth, chatter free movement.

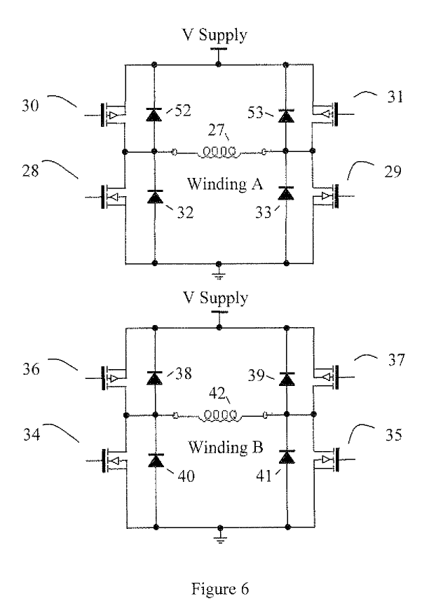

FIG. 6 shows an example of drive circuitry that can be used to drive a permanent magnet bipolar stepper motor. Bipolar stepper motors comprise two windings and have 4 wires connecting the motor to the drive electronics. A bipolar motor creates more torque than a comparable unipolar motor since current is running through the entirety of each winding (rather than ½ of the winding as in a unipolar design). In addition, a bipolar stepper motor is cheaper to produce than a comparable unipolar stepper motor. FIG. 6 shows two H drive circuits (one per winding). Current flows through winding A 27 (left to right) when MOSFETs 30 and 29 are turned on. Similarly, current flows from right to left when MOSFETS 31 and 28 are turned on. The circuit for winding B 42 mirrors that for winding A 27 (current flows left to right when 36 and 35 are on; current flows right to left when 37 and 34 are on). MOSFETs 28, 29, 34 and 35 are N-Channel MOSFETs; they require a positive bias to turn on. MOSFETs 30, 31, 36 and 37 are P-Channel MOSFETs; they require a negative bias to turn on. It is important that the MOSFETs on each side of the bridge are not simultaneously on as that will cause large current surges and potential damage to the drive circuitry. As such, the controller should be configured to carefully time the transitions during current reversals. Diodes 52, 53, 32, 33, 38, 39, 40 and 41 can be used to prevent the spikes produced by the stepper motor (back emf) from reverse biasing the MOSFETs and causing junction breakdown. There are integrated stepper motor driver circuits available in the marketplace that can be used for this type of application. They can provide the H drive circuit and protection diodes, and prevent current spikes. A person skilled in the art will be capable of employing an integrated circuit or developing a discrete design.

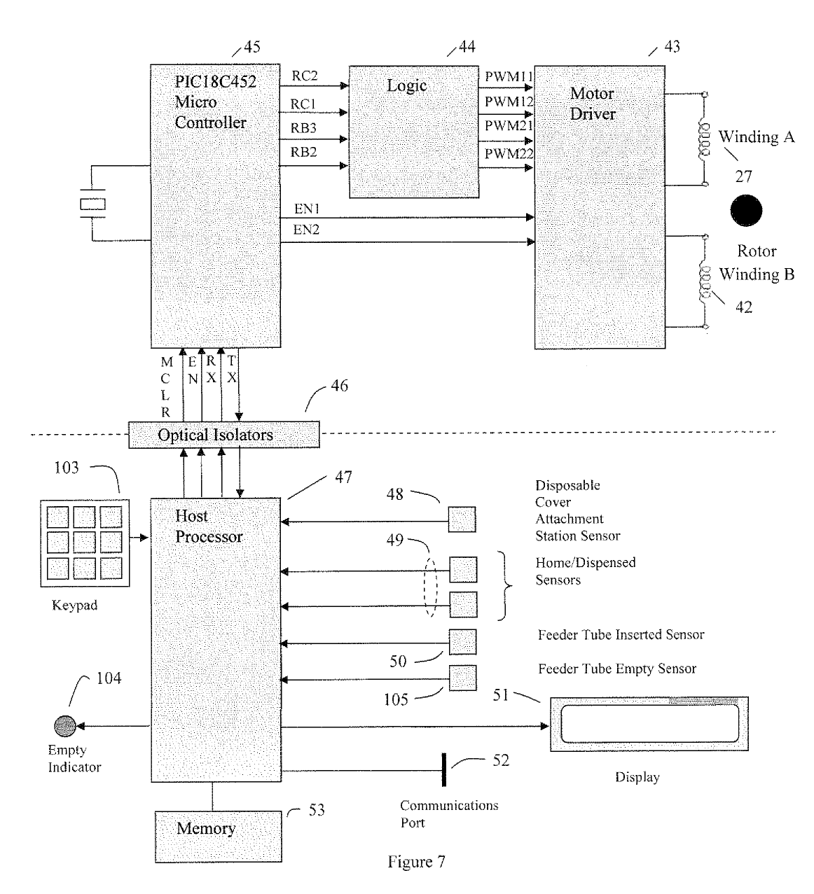

FIG. 7 is a block diagram of an embodiment of circuitry that can be used in the automated dispensing apparatus. Elements such as power supplies are not shown and a person skilled in the art will be familiar with the need and implementation of these items. A stepper motor integrated circuit 43 can be used to drive the stepper motor. It is advantageous to incorporate a drive technique called micro-stepping to drive the stepper motor. This well known technique works on the principle of gradually transitioning the current from one winding to another using pulse width modulation of the voltage across the coils of the windings A 27 and B 42, respectively. The net result is to smooth any motor jerking as it transitions between steps, reducing noise and motor resonance. The described circuitry employs micro-stepping to drive the stepper motor. The standard technique for micro-stepping a stepper motor is to use a sin-cosine algorithm to keep the net torque in the stepper motor constant. In most implementations, a lookup table is implemented in the memory of a microcontroller in order to create the correct sequence of pulse widths to apply in the voltage domain to the two coils. These algorithms are well published and available from motor vendors or controller vendors. There may be some limitations to micro-stepping due to the construction of the stepper motors and static friction. In addition, the pulse rate may be managed to prevent creating resonance in the stepper motor. The choice of the correct stepper motor and driver design is left to the designer as these design decisions are well known.

In FIG. 7, a PIC18C452 microcontroller 45 from Microchip Corporation can be used to implement the micro-stepping controller. The outputs RC2 and RC1 can be 10 bit resolution PWM controllers implemented within the microcontroller. RB3 and RB2 can select the correct inputs of the motor driver to apply the PWM signals from RC2 and RC1. The signals EN1 and EN2 can enable the motor driver to drive the windings of the stepper motor. The interface logic 44 can be a simple digital selector. The manufacturer shows the code that is required to implement the stepping function on their web page and a person skilled in the art can produce this without guidance. The microcontroller 45 can interface to the balance of the system through an isolated interface 46. This can prevent any noise associated with the motor from disrupting other system functions and ameliorate potential radiated emissions. The signals used to interface between the microcontroller 45 and a host processor 47 can include:
MCLR, which can cause the microcontroller to be reset and its code execution to begin at initialization;
EN, which can enable the microcontroller to drive the stepper motor; and
TX/RX, which is a serial interface between the host processor and the microcontroller, creating a serial communication channel between the processors.

The TX/RX serial interface can implement the control and status path between the processors. Commands issued from the host to the microcontroller can set the number of steps used in micro-stepping, set the motor rotational direction, set the motor speed, start the motor, stop the motor, single step the motor or provide a soft reset of the microcontroller. In addition, the host processor can request state from the microcontroller including operational settings. The microcontroller can provide information about operational state including any potential operational errors.

The host processor 47 is responsible for control operations of the automated dispensing apparatus. The control operations can include interfacing to a display 51, reading the state of sensors 48, 49 and 50, polling a keypad 103, setting an empty indicator 104 and instructing the microcontroller to perform the correct operation. The display 51 can be used to display the operational state of the apparatus and to display the number of disposable stethoscope head covers that have been dispensed or the number remaining in the feeder tube. In addition, it can be used to display any operational errors. A communications port 52 is shown for the purpose of remote monitoring to facilitate material management and ordering functions. The system can include sensors that can be used to detect the position of the dispensing arm 49, the presence of a disposable stethoscope head cover in the attachment station 48, the presence of the feeder tube 50 and the absence of the feeder tube 105. A system memory 53 can be used to store programs and data structures for the host processor.

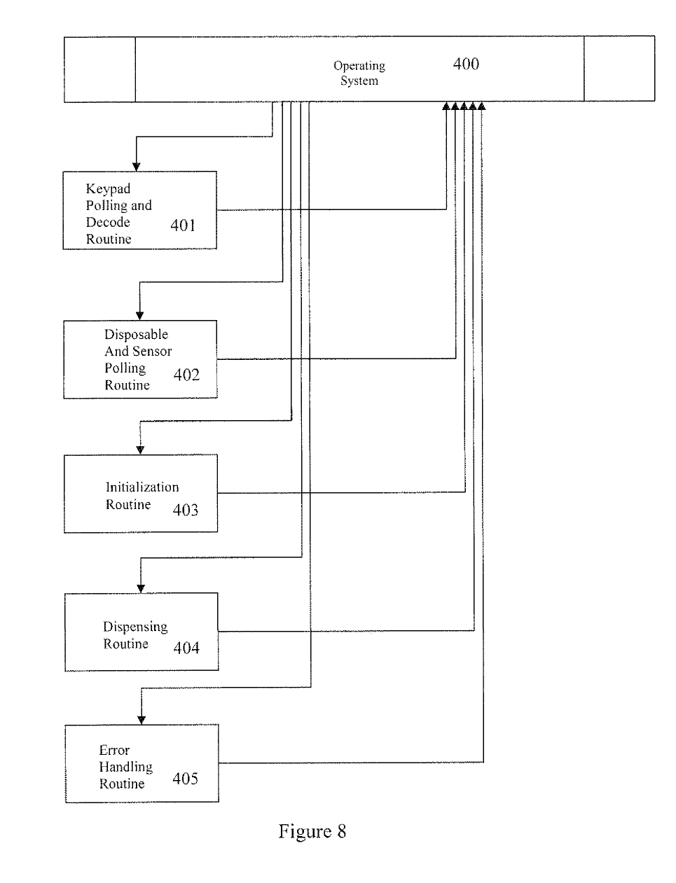

FIG. 8 shows an embodiment of top level software for use in the host processor. The host processor can run an operating system or kernel 400. There are a number of standard real time operating systems on the market including VXWorks and Linux. The operating system is responsible for memory management and scheduling the various tasks and operations that comprise the system. Several routines that can be subordinated to the operating system are shown. This is an illustrative list and the actual implementation may require more routines than shown. In addition, the timing of operations is managed, for example, wait a certain period of time after a disposable stethoscope head cover is removed from the attachment station before a new disposable stethoscope head cover is dispensed, by the individual routines (details of this are clear to a person skilled in the art).

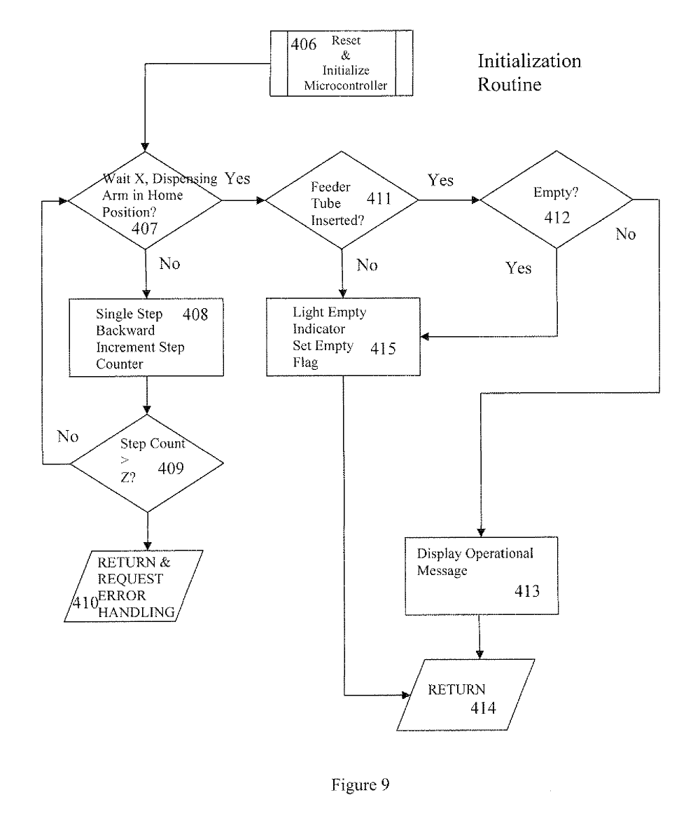

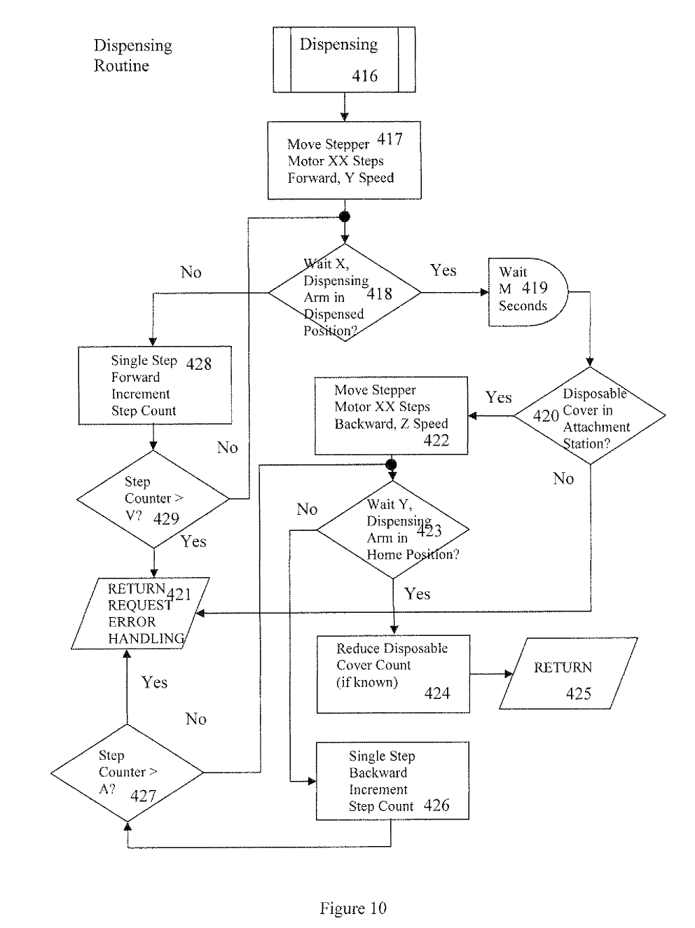

A keypad polling and decode routine 401 can be used to read the keyboard and to detect whether a key has been pressed and what the meaning of that key is to the system. A disposable and sensor polling routine 402 can be used to poll the state of the system. This can include whether a disposable stethoscope head cover is in the attachment station, whether the feeder tube is inserted and whether the feeder tube is empty. An initialization routine 403 can be used to initialize the system. It is shown in detail in FIG. 9. A dispensing routine 404 can be used to dispense a new disposable stethoscope head cover. It is shown in detail in FIG. 10. An error handling routine 405 can be used to process system errors. This can be the result of a jam, an unexpected operational result or failure of start-up or run time diagnostics (not shown).

FIG. 9 shows an example initialization routine. The system does some basic operational initialization before it is ready to go into service. The routine begins at an entry point 406 and the first order of business is to determine whether the dispensing arm is in home position 407. If it is not, then it can be single stepped 408 and 409 back until the position sensor indicates that it is in home position. If the step count 409 exceeds a certain number Z, then there is a system error as it is known that the dispensing arm should return to home position after a number of steps no matter where it is positioned. The routine can exit 410 upon detecting an abnormally large number of steps without reaching home position and request that the operating system invoke error handling 410. If the dispensing arm is in home position or is walked into home position 407, then the initialization routine can check to see whether the feeder tube is inserted 411. If it is not, then the empty light 104 (FIG. 7) can be set, the empty flag can be set 415, and control can be returned to the operating system 414. If the feeder tube is inserted then the pre-staging sensor can be checked to determine whether the feeder tube is empty 412. If the feeder tube is empty, then the empty light can be lit, the empty flag can be set 415, and control can be returned to the operating system 414. If the feeder tube is not empty, then an operational message can be displayed 413. The initialization routine can complete by returning control to the operating system 414.

FIG. 10 shows an example dispensing routine. The system can invoke this routine in response to removal of the disposable stethoscope head cover from the attachment station (automatic mode) or by manual invocation via the keypad. The routine can be entered 416 and the stepper motor can be instructed to move a number of steps forward at a particular speed 417. This can cause the dispensing arm to move a disposable stethoscope head cover from the pre-staging shelf into the attachment station. After a certain period of time (X), the dispensing arm should be in the dispensed position 418. If it is, then a short period of time can be waited 419 prior to checking that the disposable stethoscope head cover is in the attachment station 420. If it is not in the attachment station, then the routine can return with a request to invoke error handling 421. If the disposable cover is in the attachment station, then the stepper motor can be instructed to move backward a number of steps at a particular speed 422. After a certain period of time (Y), the dispensing arm is checked to see if it is in home position 423. If it is, the disposable stethoscope head cover count can be reduced 424 (if known) and the routine can return to the operating system 425. If it is not, then the stepper motor can be single stepped backwards 426 and the step counter can be checked 427 to verify that excessive steps signifying an error have not been taken. If excessive steps have been taken, then the routine can return to the operating system and request invocation of the error handling routine 421.

If the dispensing arm is not in the dispensed position 418, then the motor can be single stepped forward 428 and the overall step count can be checked 429. If the overall step count is greater than a determined number, then the routine can return to the operating system with a request for error handling 421. If the step count has not exceeded the limit, then after a short period of time, the code can check to see if the dispensing arm is in dispensed position 418.

While this invention has been particularly shown and described with references to example embodiments thereof, it will be understood by those skilled in the art that various changes in form and details may be made therein without departing from the scope of the invention encompassed by the appended claims.

What is claimed is:

1. A disposable stethoscope head cover comprising:
a hypoallergenic membrane member configured to be secured to a stethoscope head to inhibit physical contact between the skin of a patient and the stethoscope head; and
a support member configured to hold the membrane member to the stethoscope head.

2. The disposable stethoscope head cover of claim 1 wherein the hypoallergenic membrane member is made of vinyl.

3. The disposable stethoscope head cover of claim 1 wherein the support member is a fastening ring.

4. The disposable stethoscope head cover of claim 3 wherein the fastening ring comprises a perforated line facilitating removal of the disposable stethoscope head cover from the stethoscope head.

5. The disposable stethoscope head cover of claim 1 wherein the support member is made of a rigid material.

6. The disposable stethoscope head cover of claim 1 wherein the support member is configured to avoid contact with the patient during use.

7. The disposable stethoscope head cover of claim 1 further comprising a removal tab facilitating removal of the disposable stethoscope head cover from the stethoscope head.

8. An automated dispensing apparatus comprising:
a pre-staging shelf;
a feeder tube vertically disposed above the pre-staging shelf, the feeder tube configured to hold plural items wherein an item to be dispensed is fed into the pre-staging shelf;
a staging station;
a dispensing arm configured to push the item to be dispensed from the pre-staging shelf to the staging station; and
means for moving the dispensing arm.

9. The automated dispensing apparatus of claim 8 wherein the item to be dispensed is fed into the pre-staging shelf by gravity.

10. The automated dispensing apparatus of claim 8 wherein the feeder tube comprises means for forcing the item to be dispensed into the pre-staging shelf.

11. The automated dispensing apparatus of claim 10 wherein the means for forcing the item to be dispensed into the pre-staging shelf is a spring.

12. The automated dispensing apparatus of claim 8 further comprising a light source and a photo detector to detect an empty condition of the pre-staging shelf.

13. The automated dispensing apparatus of claim 8 further comprising a sensor to detect the presence of the feeder tube.

14. The automated dispensing apparatus of claim 8 further comprising a light source and a photo detector to indicate whether the item to be dispensed is positioned on the staging station.

15. The automated dispensing apparatus of claim 8 further comprising a position tab and a plurality of photo detectors to detect home and dispensed positions of the dispensing arm.

16. The automated dispensing apparatus of claim 8 further comprising a guide member wherein the dispensing arm is translated across the guide member.

17. The automated dispensing apparatus of claim 8 wherein the means for moving the dispensing arm is a wheel attachable to a linkage attachable to the dispensing arm.

18. The automated dispensing apparatus of claim 8 wherein the means for moving the dispensing arm is a drive gear.

19. The automated dispensing apparatus of claim 8 wherein the means for moving the dispensing arm is driven by a stepper motor.

20. The automated dispensing apparatus of claim 19 wherein the stepper motor is a bipolar stepper motor or a unipolar stepper motor.

21. The automated dispensing apparatus of claim 19 wherein the stepper motor is a permanent magnet bipolar stepper motor.

22. The automated dispensing apparatus of claim 19 wherein the stepper motor is driven by micro-stepping.

23. The automated dispensing apparatus of claim 22 wherein the micro-stepping is implemented by a micro controller.

24. The automated dispensing apparatus of claim 8 wherein the item to be dispensed is a disposable stethoscope head cover comprising:
a hypoallergenic membrane member configured to be secured to a stethoscope head to inhibit physical contact between the skin of a patient and the stethoscope head; and
a fastening ring configured to hold the membrane member to the stethoscope head.

25. The automated dispensing apparatus of claim 24 further comprising a recess in the center of the staging station configured to allow the stethoscope head to move downward into the recess for the disposable stethoscope head cover to snap into place.

26. The automated dispensing apparatus of claim 25 wherein the edge of the recess is configured to support the fastening ring of the disposable stethoscope head cover.

27. The automated dispensing apparatus of claim 24 further comprising a detent in the staging station configured to stop the motion of the disposable stethoscope head cover once it is pushed in the staging station.

28. The automated dispensing apparatus of claim 27 wherein the detent comprises a gap configured to allow a clinician to attach the disposable stethoscope head cover without interference with the stethoscope's acoustic tube.

29. A method of preventing cross patient exposure to infectious agents due to stethoscope carryover, the method comprising:
stretching a hypoallergenic membrane over a surface of the stethoscope head on one surface of the membrane;
holding the membrane to the stethoscope head with a support;
physically contacting a patient on the other surface of the membrane during examination with the stethoscope; and
removing the membrane and the support from the stethoscope head after examination.

30. The method of claim 29 wherein the hypoallergenic membrane is made of vinyl.

31. The method of claim 29 wherein the support is a fastening ring.

32. The method of claim 31 wherein the fastening ring comprises a perforated line facilitating removal of the membrane and the fastening ring from the stethoscope head.

33. The method of claim 29 wherein the support is made of a rigid material.

34. The method of claim 29 wherein the support does not come into contact with the patient during use.

35. The method of claim 29 wherein removal of the membrane and the support from the stethoscope head is facilitated by a removal tab.

36. A method of dispensing an item to be dispensed, said method comprising:
feeding the item to be dispensed from a feeder tube into a pre-staging shelf, and
pushing the item to be dispensed with a dispensing arm from the pre-staging shelf into position in a staging station.

37. The method of claim 36 wherein feeding comprises gravity feeding.

38. The method of claim 36 wherein feeding comprises forcing.

39. The method of claim 38 wherein forcing comprises forcing with a spring.

40. The method of claim 36 further comprising detecting an empty condition of the pre-staging shelf with a light source and a photo detector.

41. The method of claim 36 further comprising detecting the presence of the feeder tube with a sensor.

42. The method of claim 36 further comprising detecting whether the item to be dispensed is positioned on the staging station with a light source and a photo detector.

43. The method of claim 36 further comprising detecting home and dispensed positions of the dispensing arm with a position tab and a plurality of photo detectors.

44. The method of claim 36 wherein the item to be dispensed is a disposable stethoscope head cover comprising:
a hypoallergenic membrane member configured to be secured to a stethoscope head to inhibit physical contact between the skin of a patient and the stethoscope head; and
a fastening ring configured to hold the membrane member to the stethoscope head.

45. The method of claim 44 wherein the staging station comprises a recess in the center configured to allow the stethoscope head to move downward into the recess for the disposable stethoscope head cover to snap into place.

46. The method of claim 45 wherein the edge of the recess is configured to support the fastening ring of the disposable stethoscope head cover.

47. The method of claim 44 wherein the staging station comprises a detent configured to stop the motion of the disposable stethoscope head cover once it is pushed in the staging station.

48. The method of claim 47 wherein the detent comprises a gap configured to allow a clinician to attach the disposable stethoscope head cover without interference with the stethoscope's acoustic tube.
